(19) United States
(12) Patent Application Publication
(10) Pub. No.: US 2002/0107532 A1
(43) Pub. Date: Aug. 8, 2002

(54) Implantable system for anchoring stitching threads inside a bone tunnel and a kit therefor

(76) Inventors: Jacqueline Huet-Olivier, Saint-Ismier (FR); Pascal Boileau, Nice (FR)

Correspondence Address: BLANK ROME COMISKY & MCCAULEY, LLP, 900 17TH STREET, N.W., SUITE 1000, WASHINGTON, DC, 20006, US

(21) Appl. No.: 10/067,271

(22) Filed: Feb. 7, 2002

(30) Foreign Application Priority Data
Feb. 7, 2001 (FR) 01/01647

(51) Int. Cl. A61B 17/04

(52) U.S. Cl. 606/144

(57) ABSTRACT

The object of the invention is an implantable system (16) for anchoring stitching threads (2) within a bone tunnel (3), said system comprising an implantable device (1) and a complementary piece (17), the said device being formed by a single piece produced from bioresorbable material, and comprises:

a sheath (4) arranged so as to receive at least one stitching thread (2); and

a head (5) disposed at a proximal end (6) of the said sheath (4) and extending radially from the external wall (7) thereof:

the said piece being intended to be introduced into the sheath (4), so as, in a first position, to leave free the movement of the stitching threads (2) inside the sheath (4) and, in a second position, be able to block this movement.

The invention also relates to an anchoring kit comprising such a system.

The invention relates to an implantable system for anchoring suture threads within a bone tunnel and kit comprising such a system.

It applies typically to the temporary fixing of a soft tissue, for example a tendinous or muscular structure, to a bone by means of suture threads.

In a particular example, the invention applies to the repair and fixing of the rotator cuff, that is to say of the tendinous structure situated at the shoulder on the humerus of a human patient suffering from a pathology of the said cuff.

Methods are already known in which the suture threads pass through the rotator cuff before being anchored either directly by effecting points through the bone or indirectly by passing the stitch through the eyelet of an anchor previously fixed to the bone.

The fixing thus effected has a certain number of drawbacks in particular with regard to its quality.

In particular, the direct contact which exists between the stitching threads and the bone results in a “butter-cutting wire” effect, that is to say a penetration of the threads into the cortical bone bridge by shearing, which causes a relaxing of the fixing force.

This effect is all the greater, the lower the bone quality, and therefore notably in aged patients who have an osteoporotic humeral head.

Indirect fixing by anchor can also prove to be faulty by reason of the low density of the spongy bone in which it is placed.

In addition, fixing under arthroscopy is often complicated because of the difficulties in producing and clamping the knots between stitching threads.

The invention aims notably to remedy these drawbacks by providing an implantable system for anchoring stitching threads within a bone tunnel which avoids direct contact between the stitching threads and the bone whilst being easily implantable and adaptable to the different geometries of bone tunnel.

In addition, the system of the invention does not require the production of knots between stitching threads, which is well adapted for its implantation under arthroscopy, notably by means of the kit proposed.

To this end, and according to a first aspect, the invention proposes an implantable system for anchoring stitching threads within a bone tunnel, said system comprising an implantable device and a complementary piece, the said device being formed by a single piece produced from bioresorbable material, and comprises:

a sheath arranged so as to receive at least one stitching thread; and

a head disposed at a proximal end of the said sheath and extending radially from the external wall thereof;

the said piece being intended to be introduced into the sheath, so as, in a first position, to leave free the movement of the stitching threads inside the sheath and, in a second position, be able to block this movement.

According to a second aspect, the invention proposes a kit for anchoring stitching threads within a bone tunnel, comprising such an implantable system and a device for implanting the said system.

Other objects and advantages of the invention will emerge during the description which follows with reference to the accompanying drawings.

FIG. 1 depicts, in perspective, an embodiment of an implantable device.

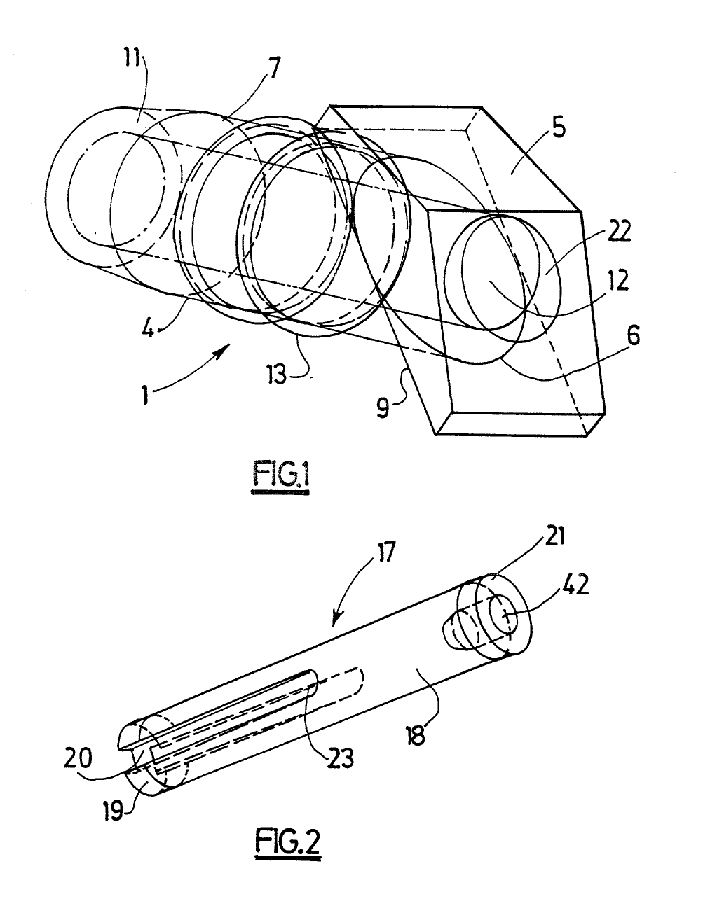

FIG. 2 depicts, in perspective, an embodiment of a complementary piece forming, with the device of FIG. 1, an implantable system.

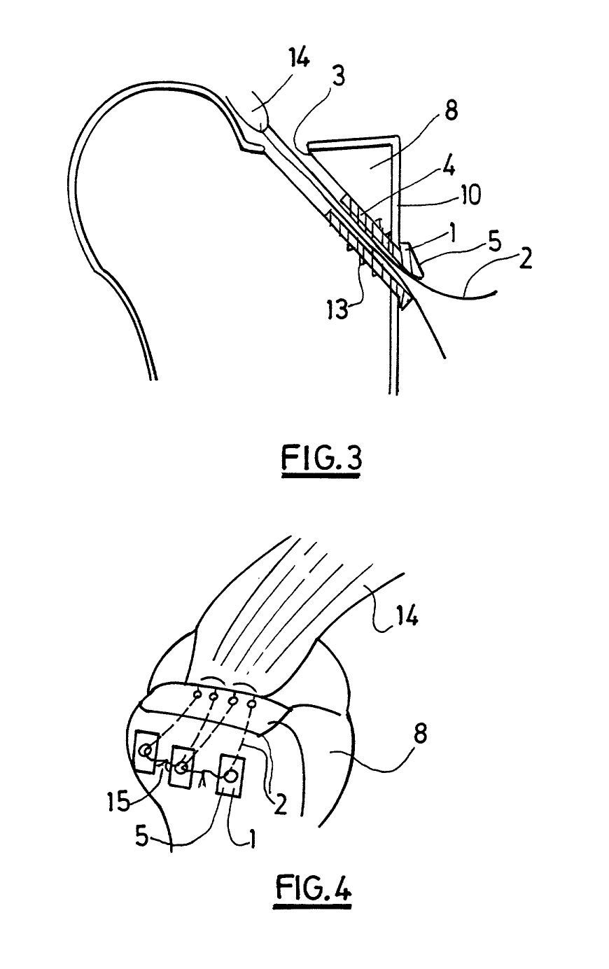

FIG. 3 depicts, in section and schematically, the device of FIG. 1 implanted in a bone tunnel.

FIG. 4 depicts, in perspective and schematically, the fixing of a rotator cuff on the humerus by means of three devices of FIG. 1, the fixing being effected by trans-device stitching points.

FIG. 5 depicts, in perspective, an implantable system comprising the device of FIG. 1 and the complementary piece of FIG. 2, before introduction of the said piece into the said device.

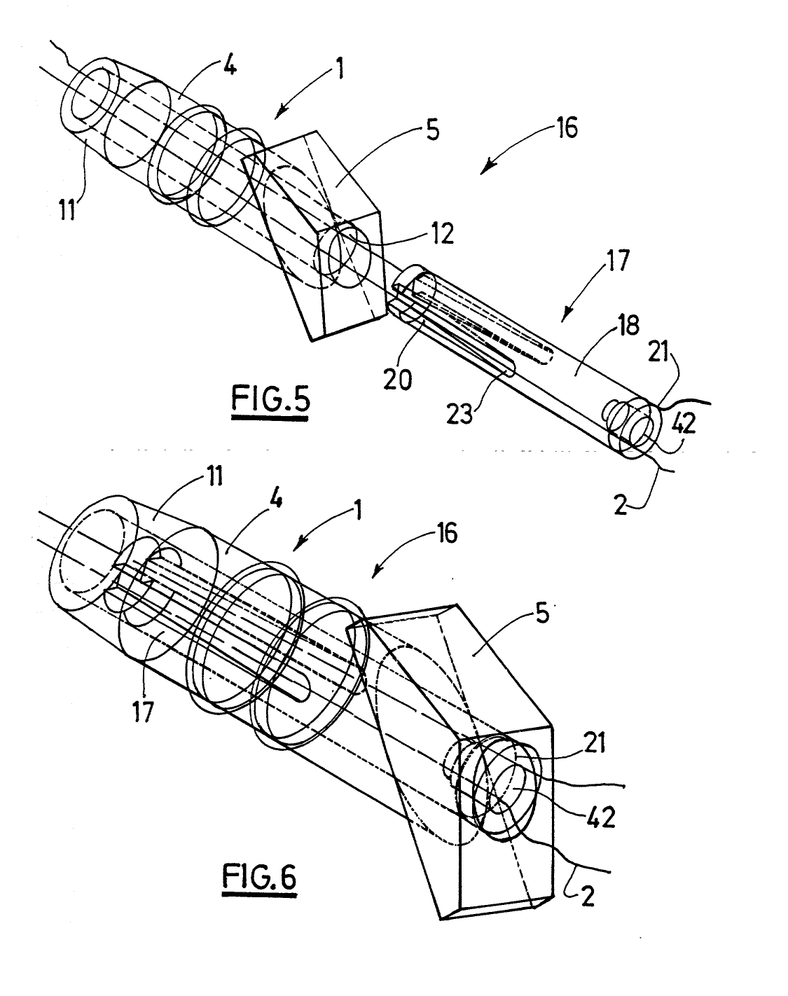

FIG. 6 depicts, in perspective, the implantable system of FIG. 6 in which the complementary piece is in its second position.

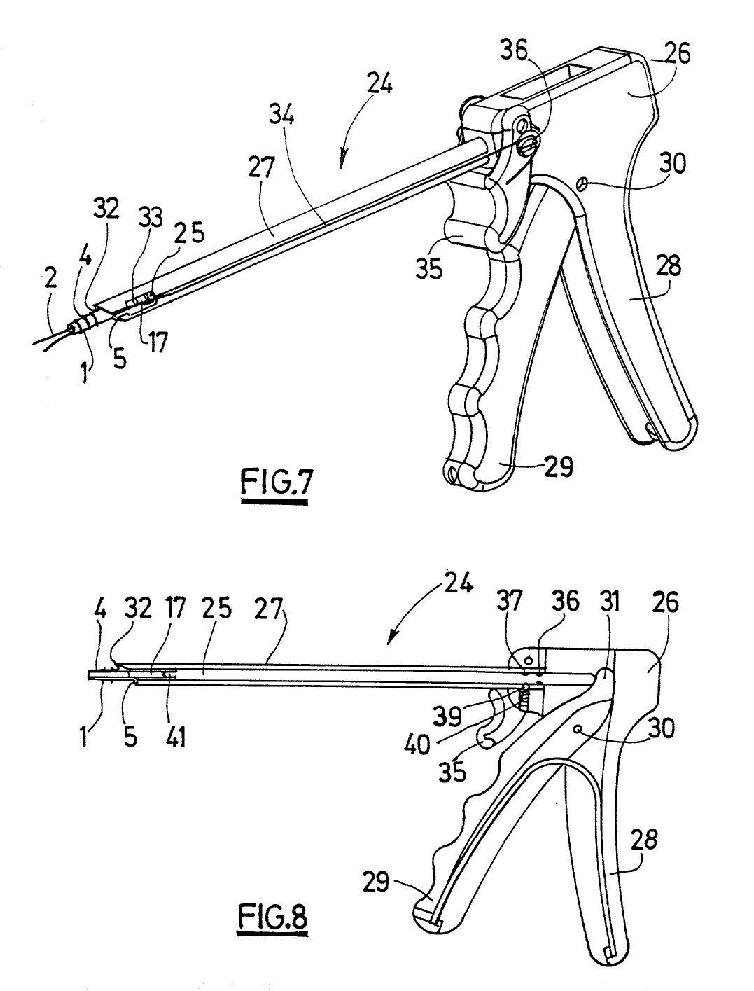

FIG. 7 depicts, in perspective, a stitching thread anchoring kit comprising the system of FIGS. 5 and 6 and a device for implanting the said system, the said system being mounted on the device to allow its implantation.

FIG. 8 is a longitudinal section of the kit depicted in FIG. 7.

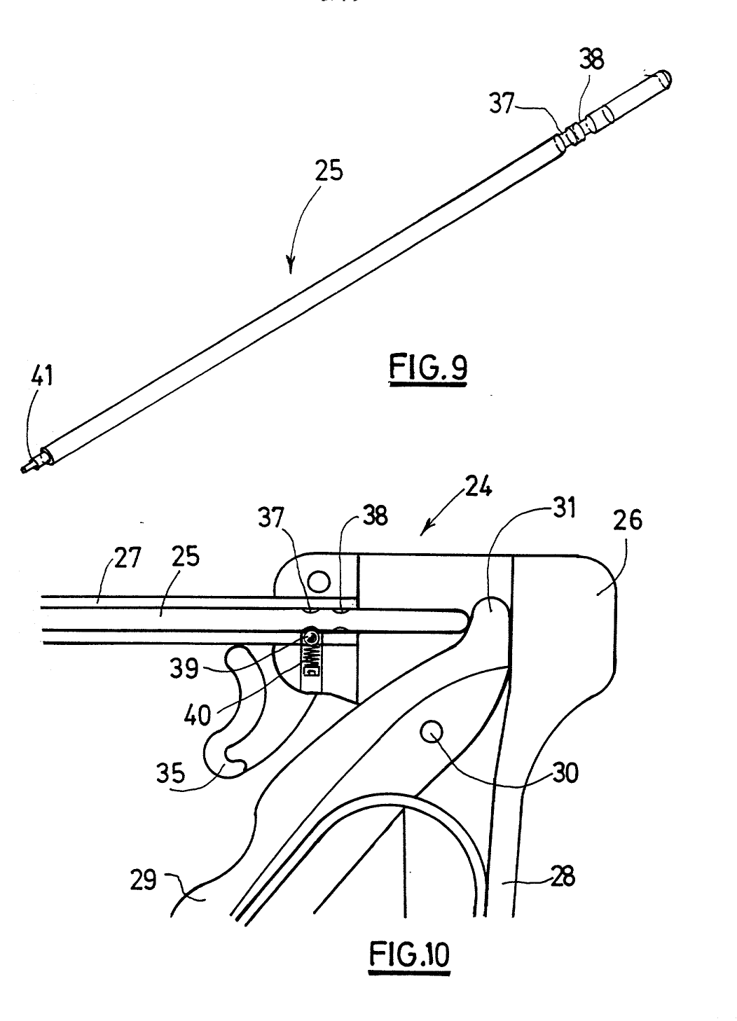

FIG. 9 depicts, in perspective, a piece for actuating the complementary piece.

FIG. 10 depicts, in section and partially, the device of the piece in FIG. 9 in the implantation device in FIGS. 7 and 8.

In relation to FIG. 1, an implantable device 1 is described for anchoring stitching threads 2 inside a bone tunnel 3 which comprises:

a sheath 4 arranged to receive at least one stitching thread 2; and

a head 5 disposed at a proximal end 6 of the said sheath and extending radially from the external wall 7 thereof.

In the description the terms “proximal” and “distal” are defined with respect to the direction of implantation of the device 1 in the bone tunnel 3.

The device 1 is formed by a bioresorbable material and is produced in a single piece, notably by injection moulding.

According to one embodiment, so that it has good suitability for moulding, the bioresorbable material comprises high chemical purity polymers, with a molecular mass greater than approximately 250,000 and of low polydispersity, for example less than 2.

The bioresorbable material can comprise the stereocopolymers of L-and D-lactic acid, the homopolymers of L-lactic acid, the copolymers of lactic acid and a compatible comonomer such as the derivatives of alpha-hydroxy acids, as well as the derivatives and/or mixtures of these substances, for example synthesised according to the method described in the patent application FR-2 745 005 issuing from the applicant.

In a particular example, the bioresorbable material comprises 98% L-lactic acid and 2% D-lactic, its mean molecular mass is between 300,000 and 400,000 and its polydispersity index is 1.8.

Because of the arrangement of the stitching threads 2 in the sheath 4 and the presence of the head 5 extending radially at the area where the clamping force is the highest, the device 1 prevents destructive contacts between the threads 2 and the bone 8.

Moreover, the head 5, because of the different angles which its lower surface 9 (that is to say the face intended to come into contact with the bone 8) can form with the sheath 4, makes it possible to adapt the device 1 to the different geometries of the bone tunnel 3.

This is because the angle formed between the bone tunnel 3 previously pierced and the surface of the bony cortical 10 can be variable according to the geometric constraints of the place of implantation and, for good stability of the device 1, the lower surface 9 of the head 5 must come into quasi-plane contact with the bone surface 10.

In a first example (see FIG. 3) and when this angle is substantially 45°, the device 1 can be produced so that the lower face 9 of the head 5 forms an angle of substantially 45° with the longitudinal axis of the sheath 4.

In a second example (not shown) and when this angle is substantially 90°, the device 1 can be produced so that the lower face 9 of the head 5 forms an angle of substantially 90° with the longitudinal axis of the sheath 4.

In addition, the head 5 can be rectangular in shape (see figures) in order to limit the bulk of the device 1 on the bone 8 whilst ensuring good stability of the implant.

As a variant and to allow better fixing between the device 1 and the bone 8 in which it is implanted, the external wall 7 of the sheath 4 comprises means of anchoring the device 1 in the bone tunnel 3.

According to one embodiment (see FIG. 1), the sheath 4 is formed by an annular piece with an outside diameter substantially equal to or slightly less than that of the bone tunnel 3 and whose distal end 8 is frustoconical in shape so as to facilitate its insertion into the bone tunnel 3.

In addition, the piece 4 has an opening 12 with a substantially circular cross-section which passes right through along its longitudinal axis so as to have the stitching threads 2 pass through it.

The anchoring means can then be formed by at least one protrusion 13 in the form of a ring which extends radially on the external surface 7 of the annular piece and whose outside diameter is substantially equal to or slightly greater than that of the bone tunnel 3.

At the time of implantation (see FIG. 3), the device 1 is forcibly introduced into the bone tunnel 1 until the lower surface 9 of the head 5 comes into quasi-plane contact with the surface 10 of the cortical bone. The stitching threads 2 are then received in the sheath 4 of the device I so as to be able to slide freely.

FIG. 4 depicts the fixing of a rotator cuff 14 on the humerus 8 by means of three devices 1 implanted in three different bone tunnels 3. In this example, two stitching threads 2 are previously associated with the rotator cuff 14 with four free ends. The two external ends are respectively disposed in the two external devices 1 and the other two ends are disposed in the central device 1, and the fixing is then effected by trans-device fixing points 15 between adjacent stitching thread ends 2.

A description is given below of one embodiment of an implantable system 16 comprising a device 1 as described above and a complementary piece 17 which locks the movement of the stitching threads 2 inside the sheath 4 so as to dispense with the production of stitching points 15.

The complementary piece 17 is intended to be introduced into the sheath 4 so that, in a first position, it leaves free the movement of the stitching threads 2 inside the sheath 4 and, in a second position, it is able to block this movement.

Thus, in the first position, the stitching threads 2 can be tensioned in order to provide good contact between the rotator cuff 14 and the bone 8 and then, in the second position, the stitching threads 2 are locked in position without having recourse to points 15.

According to one embodiment (see FIG. 2) the complementary piece 17 is formed by a cylindrical rod 18 whose cross-section is slightly less than the inside diameter of the sheath 4 and whose distal part 19 is provided with grooves 20 intended each to receive a stitching thread 2, the said grooves 20 being arranged on the one hand to separate the said threads 2 and on the other hand to guide their sliding inside the sheath 4.

To this end, the depth of the grooves 20 is designed to be greater than the diameter of the stitching threads 2.

In a particular example, the distal part 19 comprises as many grooves 20 as there are threads 2 intended to be received in the sheath 4, namely two grooves 20 in the embodiment depicted in FIG. 2, the said grooves 20 extending in the longitudinal direction and over less than half the total length of the rod 18 so as to obtain a good compromise between the sliding and locking of the threads 2.

In addition, a head 21 can be provided on the proximal end of the rod 18, the said head 21 being intended on the one hand to participate in the wedging of the threads 2 when the rod 18 is in its second position and on the other hand to serve as a stop when the rod 18 is positioned in the said second position.

To allow good complementarity between the device 1 and the complementary piece 17, the proximal opening 22 in the sheath 4 and the head 21 of the rod 18 are conical in shape, the diameter of the head 21 being greater than that of the proximal opening 22, the angulation of the head 21 being substantially equal to or greater than that of the proximal opening 22.

In addition, the height of the head 21 can be designed to be substantially equal to or less than the depth of the cone of the proximal opening 22, the head 21, when the piece 17 is in its second position, thus not projecting beyond the device 1 in order not to risk “injuring” the surrounding environment.

In a particular example, the angulations of the head 21 and of the proximal opening 22 are around 10°.

The complementary piece 17 is also made from bioresorbable material, identical or not to that forming the device 1.

A description is given below, in relation to FIGS. 5 and 6, of the association of the complementary piece 17 depicted in FIG. 2 in the device 1 depicted in FIG. 1.

The piece 17 is inserted in the sheath 4 with the two stitching threads 2 disposed respectively in a groove 20 so as on the one hand to separate the threads 2 well and on the other hand to allow easy sliding of the piece 17 in the sheath 4.

The piece 17 is then inserted partially in the sheath 4 and then the threads 2 are tensioned so as to obtain good contact between the rotator cuff 14 and the bone 8.

When this good contact is established, the piece 17 is forcibly pushed into the sheath 4 in order to block the movement of the threads 2. The wedging is effected on the one hand between the inclined faces 23 of the grooves 20 and the internal surface of the sheath 4 and on the other hand between the conical surfaces of respectively the head 21 and the opening 22.

This design makes it possible to obtain an effective locking of the threads 2 so that, when a tension force is applied to the threads 2, the head 21 serves as a stop for the translational movement of the piece 17 within the sheath 4.

A description is given below of a kit for anchoring stitching threads inside a bone tunnel 3 which comprises an implantable system 16 as described above and an implantation device 24 for the said system 16.

According to the embodiment depicted in the figures, the implantation device 24 comprises:

a piece 25 for actuating the complementary piece 17 from its first position to its second position; and

a tool 26 arranged to receive the implantable device 1, the stitching threads 2, the complementary piece 17 and the actuation piece 25, the said tool 26 comprising means of actuating the actuation piece 25.

The actuation piece 25 and the tool 26 can be produced from metallic or polymeric material.

When the complementary piece 17 is in its first position, the fitting of the system 16 within the bone tunnel 3 is effected by causing it to slide on the stitching threads 2 to be fixed and then the locking of the stitching threads 2 is effected by actuating the complementary piece 17.

In the embodiment depicted in the figures, the tool 26 comprises a tube 27 in which the actuation piece 25 is disposed slidably, a handle 28 and a first trigger 29 making it possible to cause the movement of the actuation piece 25 inside the tube 27 on a controlled travel.

The first trigger 29, mounted so as to pivot about a spindle 30, comprises means 31 able to push the pieces 17, 25 on a controlled travel inside the tube 27.

The distal end 32 of the tube 27 corresponds substantially to the impression of the head 5 of the implantable device 1 so as to be able to house it.

To allow on the one hand the fitting and the passage of the stitching threads 2 and on the other hand to immobilise the implantable device 1 by snapping on, the distal end 32 of the tube 27 comprises two slots 33 disposed on each side of the tube 27.

Two grooves 34 are also provided on the external face of the tube 27 and in line with the two slots 33 so as to guide the stitching 2 inside the tube 27.

A thread manipulation device is provided close to the proximal end of the tube 27 which comprises:

a first trigger 35 arranged on the handle 28 so as to be able to actuate it conjointly with the first 29; and

two lugs 36 disposed on each side of the handle 28 to allow the respective locking of a stitching thread 2 by winding.

Thus a controlled traction on the threads 2 can be exerted by the operator by actuating the second trigger 35 so as, prior to the actuation of the piece 25, to ensure good contact between the rotator cuff 14 and the bone 8.

In relation to FIG. 9, a description is given of an actuation piece 25 with a cylindrical shape, whose diameter is substantially less than the inside diameter of the tube 25 and which comprises two grooves 37, 38 machined radially on its surface close to its proximal end.

The grooves 37, 38 are arranged to cooperate with a system for locking the translation of the actuation piece 25 inside the tube 27.

The system comprises a pin 39 mounted on a spring 40 which is disposed in the handle 28 perpendicular to the movement of the actuation piece 25 so as to come to be engaged in one of the grooves 37, 38 in order to lock the translation (see FIG. 10).

Thus, when the pin 38 is engaged in the first groove 37, the threads 2 can be disposed inside the sheath 4 whilst the piece 14 is already partially engaged in the sheath 4 and then, by partial actuation of the first trigger 29, the pin 39 is engaged in the second groove 38 so as to put the piece 17 in its first position.

The operator can then actuate the second trigger 35 in order to tension the threads 2 and then completely actuate the first trigger 29 in order to put the piece 17 in its second position.

The distal end 41 of the actuation piece 25 can be threaded in order to be able, prior to its arrangement in the tool 26, to associate with it the complementary piece 17 by threading the inside of the head 21 previously provided with a hole 42.

Thus, in the event of faulty handling, the piece 17 can easily be disengaged from the sheath 4.

In this embodiment, the complementary piece 17 can be mounted on the actuation piece 25 prior to its introduction into the tube 27 and a device (not shown) makes it possible to disengage the actuation piece 25 from the complementary piece 17 in its second position, for example by providing, at the proximal end of the actuation piece 25, a knurled wheel able to rotate it.

1. An implantable system (16) for anchoring stitching threads (2) within a bone tunnel (3), characterised in that it comprises an implantable device (1) and a complementary piece (17), the said device being formed by a single piece produced from bioresorbable material, and comprises:
a sheath (4) arranged so as to receive at least one stitching thread (2); and
a head (5) disposed at a proximal end (6) of the said sheath (4) and extending radially from the external wall (7) thereof;
the said piece being intended to be introduced into the sheath (4), so as, in a first position, to leave free the movement of the stitching threads (2) inside the sheath (4) and, in a second position, be able to block this movement.

2. A system according to claim 1, characterised in that the external wall (7) of the sheath (4) comprises means of anchoring the device (1) in the bone tunnel (3).

3. A system according to claim 1 or 2, characterised in that the sheath (4) is formed by an annular piece whose distal end (11) is frustoconical in shape, the said piece having an opening (12) with a substantially circular cross-section which passes right through on its longitudinal axis.

4. A system according to claim 3, characterised in that the anchoring means are formed by at least one protrusion (13) in the form of a ring which extends radially on the external surface (7) of the annular piece.

5. A system according to any one of claims 1 to 4, characterised in that the head (5) has a rectangular cross-section, the lower face (9) of the said head (5) forming an angle of substantially 45° with the longitudinal axis of the sheath (4).

6. A system according to any one of claims 1 to 4, characterised in that the head (5) is rectangular in cross-section, the lower face (9) of the said head (5) forming an angle of substantially 90° with the longitudinal axis of the sheath (4).

7. A system according to any one of claims 1 to 6, characterised in that the complementary piece (17) is formed by a cylindrical rod (18) whose cross-section is slightly less than the inside diameter of the sheath (4) and the distal part (19) of which is provided in the grooves (20) each intended to receive a stitching thread (2), the said grooves (20) being arranged so as on the one hand to separate the said threads (2) and on the other hand to guide their sliding inside the sheath (4).

8. A system according to claim 7, characterised in that the distal part (19) comprises two grooves (20) extending in the longitudinal direction and over at least half of the total length of the rod (18).

9. A system according to any one of claims 1 to 8, characterised in that a head (21) is provided on the proximal end of the rod (18), the said head (21) being intended on the one hand to participate in the wedging of the threads (2) when the rod (18) is in its second position and on the other hand to serve as a stop during the positioning of the rod (18) in the said second position.

10. A system according to claim 9, characterised in that the proximal opening (22) in the sheath (4) and the head (21) of the rod (18) are conical in shape, the diameter of the head (21) being greater than that of the proximal opening (22), the angulation of the head (21) being substantially equal to or greater than that of the proximal opening (22) and the height of the head (21) being substantially equal to or less than the depth of the cone of the proximal opening (22).

11. A system according to claim 10, characterised in that the angulations of the head (21) and of the proximal opening (22) are around 10°.

12. A system according to any one of claims 1 to 11, characterised in that the device (1) and the complementary piece (17) are made from bioresorbable material, identical or not.

13. A system according to claim 12, characterised in that the bioresorbable material comprises the polymers of high chemical purity, with a molecular mass greater than approximately 250,000 and of low polydispersity, for example less than 2.

14. A system according to claim 13, characterised in that the bioresorbable material comprises the stereocopolymers of L- and D-lactic acids, the homopolymers of L-lactic acid, the copolymers of lactic acid and a compatible comonomer such as the derivatives of alphahydroxy acids, and the derivatives and/or mixtures of these substances.

15. A kit for anchoring stitching threads (2) inside a bone tunnel (3), comprising an implantable system (16) according to any one of claims 1 to 14 and a device (24) for implanting the said system (16).

16. A kit according to claim 15, characterised in that the implantation device (24) comprises:
a piece (25) for actuating the complementary piece (17) from its first position to its second position;
a tool (18) arranged to receive the implantable device (1), the stitching threads (2), the complementary piece (17) and the actuation piece (25), the said tool (18) comprising means of actuating the actuation piece (25);
so as to allow, when the complementary piece (17) is in its first position, the fitting of the system (16) within the bone tunnel (3) by causing it to slide on the stitching threads (2) to be fixed and then the locking of the stitching threads (2) by actuation of the complementary piece (17).

17. A kit according to claim 16, characterised in that the tool (26) comprises a tube (27) in which the actuation piece (25) is slidably disposed, a handle (28) and a first trigger (29) making it possible to cause the movement of the actuation piece (25) within the tube (27) on a controlled channel.

18. A kit according to claim 17, characterised in that the shape of the distal end (32) of the tube (27) corresponds substantially to the impression of the head (25) of the implantable device (1) so as to be able to house it.

19. A kit according to claim 18, characterised in that the distal end (32) of the tube (27) also comprises two slots (33) disposed on each side of the tube (27), the said slots (33) being arranged so as to allow on the one hand the fitting and passage of the stitching threads (2) and on the other hand to immobilise the implantable device (1) by snapping on.

20. A kit according to claim 19, characterised in that two grooves (34) are provided on the external face of the tube (27) and in line with the two slots (33) so as to guide the stitching threads (2) outside the tube (27), a device for manipulating the threads (2) being provided close to the proximal end of the tube (27).

21. A kit according to claim 20, characterised in that the manipulation device comprises:
a second trigger (35) arranged on the handle (28) so as to be able to be actuated conjointly with the first (29);
two lugs (36) disposed on each side of the handle (28) to allow the respective locking of a stitching thread (2) by winding;
so as to be able to exert a controlled traction on the threads (2) by actuation of the second trigger (35).

22. A kit according to any one of claims 17 to 21, characterised in that the actuation piece (25) is cylindrical in shape, the diameter of which is substantially less than the inside diameter of the tube (27) and comprises two grooves (37, 38) machined radially on its surface close to its proximal end, the said grooves (37, 38) being arranged to cooperate with a system for locking the translation of the actuation piece (25) inside the tube (27), the said system comprising a pin (39) mounted on a spring (40) which is disposed in the handle (28) perpendicular to the movement of the actuation piece (25) so as to come to be engaged in one of the grooves (37, 38) in order to lock the translation.

23. A kit according to any one of claims 16 to 22, characterised in that the distal end (41) of the actuation piece (25) is threaded so as to be able, prior to its arrangement in the tool (26), to associate with it the complementary piece (17) by threading the inside of the head (21) with a hole previously made in it.
